(19) United States
(12) Patent Application Publication
(10) Pub. No.: US 2013/0280187 A1
(43) Pub. Date: Oct. 24, 2013

(54) Compositions and Methods for Improving the Appearance of Facial Pores

(71) Applicant: THE PROCTER & GAMBLE COMPANY, Cincinnati, OH (US)

(72) Inventors: Rosemarie NMN OSBORNE, Oxford, OH (US); Lisa Ann MULLINS, West Chester, OH (US); Gregory Joseph KRAMER, Lawrenceburg, IN (US)

(73) Assignee: The Procter & Gamble Company, Cincinnati, OH (US)

(21) Appl. No.: 13/863,608

(22) Filed: Apr. 16, 2013

Related U.S. Application Data
(60) Provisional application No. 61/636,185, filed on Apr. 20, 2012.

(51) Int. Cl. A61K 8/97; A61Q 19/00

(52) U.S. Cl. CPC A61K 8/97; A61Q 19/00
USPC 424/59; 424/757

(57) ABSTRACT

A method of improving the appearance of facial pores is provided. The method includes the step of applying a composition having an effective amount of carob fruit extract to an area of facial pores, wherein the composition is applied for a period of time sufficient for the material to improve the appearance of the facial pores. The method may also include the step of identifying facial pores on a facial skin surface.

### TECHNICAL FIELD

Provided herein are compositions and methods for improving the appearance of facial pores using carob fruit extract.

### BACKGROUND

The epidermis, the outermost layer of the skin, comprises a cellular continuum of four layers: the stratum corneum, the granular layer, the spinous layer, and the basal layer. Each cellular layer in the epidermis represents various stages along a process in which basal epidermal keratinocytes undergo a continuous cycle of proliferation, differentiation, and apoptosis, moving upward from the basal layer to finally yield corneocytes. These corneocytes form the cornified layer known as the stratum corneum.

Basal keratinocytes reside at the lower portion of the epidermis. These mitotically active cells undergo a proliferative cycle to generate daughter cells that are physically dislocated upward into the spinous and granular layers and undergo the process of differentiation into corneocytes. On passing through the spinous and granular layers, the cells undergo morphological changes that render them flatter in structure as they lose their cellular viability, undergo alternate keratin expression profiles, and transform into cellular remnants. On average, a younger-aged epidermis turns over in about one month, shedding the older cells and replacing them with newer ones, but this process can increase to over forty days in older skin.

The stratum corneum's corneocytes remain connected to one another via proteins and lipids, creating a protective barrier between the organism and its outside environment. This tightly regulated epidermal permeability barrier functions as a physical and selective barrier against chemical and biological insults. Important functions of this barrier include attenuation of the penetration of free radicals and prevention of penetration of harmful radiation, including UV radiation, into deeper layers. The stratum corneum also acts as a permeability barrier and functions to prevent loss of body moisture to the outside environment. Dysfunction of this barrier can lead to chronic skin conditions, diseases, and in extreme cases can even threaten the viability of the organism.

Skin aging is a multifactorial process driven by both intrinsic (chronological aging) and extrinsic (environmental) factors, including ultraviolet (UV) exposure, environmental toxins, pollutants, and smoking. It is well known in the art that the ability of the stratum corneum to cyclically generate new layers of skin diminishes with age so that the stratum corneum turnover rate is substantially reduced in aged skin, with the cornified layer becoming gradually thinner. This results in a reduction in the functioning capacity of the barrier so that harmful stimuli penetrate the stratum corneum more easily, leading to UV-damage, for example, of the underlying dermal layers, degradation of collagen and elastin, and eventually manifests in appearance as wrinkling and skin atrophy. Further, the barrier suffers from an age-related increase in permeability to free radicals and a reduction in the amount of lipid in the intercellular matrix, decreasing barrier capacity to diffuse toxins from deeper layers. Recovery capacity of the barrier to environmental insult is also substantially reduced with age.

Over time, these processes can result in the appearance of enlarged facial skin pores, particularly around the nose and cheeks. While the age at which facial pores may begin to enlarge can vary widely from individual to individual, the process generally begins around age twenty and the pores may continue to enlarge and become more defined between the ages of forty and fifty. Accordingly, it would be desirable to provide topically applied cosmetic compositions and associated methods of treatment that improve the appearance of enlarged facial pores.

### SUMMARY OF THE INVENTION

In order to provide a solution to the aforementioned problems, there is provided a method of improving the appearance of facial pores, which comprises applying a composition with an effective amount of carob fruit extract to an area of facial skin having facial pores. The carob-containing composition is applied for a period of time sufficient for the carob fruit extract to improve the appearance of the facial pores. In some instances, it may be desirable to first identify a region of facial pores on a facial skin surface in need of treatment.

### DETAILED DESCRIPTION OF THE INVENTION

All percentages and ratios used herein are by weight of the total composition and all measurements made are at 25° C., unless otherwise designated. All numeric ranges are inclusive of narrower ranges; delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

The term “apply” or “application” as used in reference to a composition, means to topically apply or spread the compositions of the present invention onto an external human skin surface such as the epidermis.

The term “dermatologically acceptable” as used herein means that the compositions or components described are suitable for use in contact with human skin tissue without undue toxicity, incompatibility, instability, allergic response, and the like.

The term “effective amount” as used herein means an amount of a compound or composition sufficient to significantly induce a positive benefit. In the present context, “effective amount” refers to improvement in the appearance of enlarged facial pores.

The term “facial pores” when used in reference to human facial skin refers generally to facial pores visible to the naked eye. A facial pore includes both the pore opening and the skin immediately adjacent to the opening that affects the visible appearance of the pore. Facial pores generally have a circular or elliptical shape at the skin surface, and generally have a pore area less than 2.0 mm².

The term “facial skin” as used herein refers to one or more of forehead, periorbital, cheek, perioral, chin, and nose skin surfaces.

The term “improving” when used in reference to facial pores includes preventing, delaying, and/or reducing the appearance of enlarged facial pores. “Improving” also thus includes decreasing the diameter of the pore opening and/or improving the appearance of the skin immediately adjacent the pore opening so that the overall appearance of the pore is reduced; this can be evaluated through quantitative (e.g., an imaging device) and/or qualitative means (e.g., visual inspection by the human eye).

### I. Compositions

The topical compositions suitable for use herein may be provided in a wide variety of product forms known in the art, e.g., solutions, suspensions, lotions, creams, gels, toners, sticks, pencil, sprays, aerosols, ointments, cleansing liquid washes and solid bars, shampoos and hair conditioners, pastes, foams, powders, mousses, shaving creams, wipes, strips, patches, electrically-powered patches, wound dressing and adhesive bandages, hydrogels, film-forming products, facial and skin masks (with and without insoluble sheet), make-up such as foundations, eye liners, and eye shadows, and the like. The composition form may follow from the particular dermatologically acceptable carrier chosen, if present in the composition.

A. Carob Fruit Extract

The compositions herein comprise an effective amount of carob fruit extract. The amount of extract that is “effective” can differ from one particular source (e.g., manufacturer) of extract to another, and can be determined by the skilled artisan based upon the particular extract product's level of activity (e.g., level of active components present). As with any extract, the concentration of active components in the particular extract product to be used will depend on factors such as the final dilution volume of the extract product, the particular extraction method employed, the natural range of variation among individual plants, and other common factors known to those skilled in the art.

The carob fruit extract (INCI name: Ceratonia siliqua fruit extract; CAS Number: 84961-45-5) of the present invention is made from the oblong, non-fleshy, bean-like pod that grows on the carob tree, which belongs to the legume family Fabaceae. Carob is rich in oligogalactomannans, which are believed to be important biological actives. The carob fruit pod contains large seeds commonly referred to as “carob nuts”.

Carob fruit extract can be derived from the fruit pod, the seeds, or combinations thereof, using processes known in the art. The carob fruit extract may include other suitable materials such as, for example, water, thickeners, humectants, solvents, solubilizers, etc. A suitable carob fruit extract for use herein is commercially produced by Silab S.A. (France), under the trade name Glyco-Repair™PX. This particular extract product contains approximately 94% water, 5% carob fruit extract, and 1% other materials.

In some embodiments, the composition comprises the carob fruit extract in an amount of from 0.0001% to 15%, from 0.0002% to 10%, from 0.001% to 15%, or even from 0.025% to 10%. In other embodiments, the composition may include carob fruit extract in an amount of from 0.05% to 10%, in others from 0.05% to 5%, and in others from 0.1% to 5%, by weight of the total composition.

B. Optional Components

The compositions herein may contain a variety of other ingredients provided that they do not unacceptably alter the benefits of the invention. When present, compositions herein may contain from about 0.0001% to about 50%; from about 0.001% to about 20%; or, alternately, from about 0.01% to about 10%, by weight of the composition, of the optional components. The amounts listed herein are only to be used as a guide, as the optimum amount of the optional components used in a composition will depend on the specific active selected since their potency does vary considerably. Hence, the amount of some optional components useful in the present invention may be outside the ranges listed herein.

The optional components, when incorporated into the composition, should be suitable for use in contact with human skin tissue without undue toxicity, incompatibility, instability, allergic response, and the like. The compositions of the present invention may include optional components such as anti-acne actives, desquamation actives, anti-cellulite agents, chelating agents, flavonoids, tanning active, non-vitamin antioxidants and radical scavengers, hair growth regulators, anti-wrinkle actives, anti-atrophy actives, minerals, phytosterols and/or plant hormones, N-acyl amino acid compounds, antimicrobial or antifungal actives, and other useful skin care actives, which are described in further detail in U.S. application publication No. US2006/0275237A1 and US2004/0175347A1.

The Personal Care Product Council's International Cosmetic Ingredient Dictionary and Handbook, Thirteenth Edition, describes a wide variety of non-limiting cosmetic and pharmaceutical ingredients commonly used in the skin care industry, which are suitable optional components for use in the compositions of the present invention. Examples of these ingredient classes include: abrasives, absorbents, aesthetic components such as fragrances, pigments, colorings/colorants, essential oils, anti-caking agents, antifoaming agents, antimicrobials, binders, biological additives, buffering agents, bulking agents, chelating agents, chemical additives, colorants, cosmetic astringents, cosmetic biocides, denaturants, drug astringents, emollients, external analgesics, film formers or materials, opacifying agents, pH adjusters, preservatives, propellants, reducing agents, sequestrants, skin cooling agents, skin protectants, thickeners viscosity modifiers, vitamins, and combinations thereof.

Several classes of optional ingredients are discussed in more detail below.

1. Skin Tone Agents

In some embodiments, it may be desirable to include a skin tone agent in the composition in combination with the carob fruit extract. As used herein, “skin tone” refers to generalized areas and/or regionalized areas (i.e. spots, age spots) of hyperpigmentation. As used herein, “improving the skin tone” means preventing or reducing the appearance of hyperpigmented areas.

The skin tone agents can be included to further improve overall skin tone. When present, the compositions herein may contain up to about 50%, 40%, 30%, 20%, 10%, 5%, or 3% by weight, based on the weight of the composition, of the skin tone agent. When present, the compositions herein may contain at least 0.001%, 0.01%, 0.1%, 0.2%, 0.5%, or 1%, by weight, based on the weight of the composition, of the skin tone agent. Suitable ranges include any combination of the lower and upper limits including suitable ranges from about 0.1% to about 50%; from about 0.2% to about 20%; or from about 1% to about 10%, by weight of the composition, of the skin tone agent. The amounts listed herein are only to be used as a guide, as the optimum amount of the skin tone agent will depend on the specific active selected since their potency does vary considerably.

Suitable skin tone agents include, but are not limited to, sugar amines, vitamin B3 compounds, arbutin, deoxyarbutin, 1,3-dihydroxy-4-alkylbenzene such as hexylresorcinol, bakuchoil (4-[(1E,3S)-3-ethenyl-3,7-dimethyl-1,6 octadienyl]phenol or monterpene phenol), pyrenoine (available from Biotech Marine, France), panicum miliaceum seed extract, arlatone dioic acid, cinnamic acid, ferulic acid, achromaxyl, methyl nicotinamide, oil soluble licorice extract, folic acid, undecylenic acid (i.e., undecenoic acid), zinc undecylenate, thiamine (Vitamin B1) and its hydrochloride, L-tryptophan, ficus benghalensis, phlorogine (laminaria) helianthus annuus (sunflower) and vitis vinifera (grape) leaf extract, carnosine (i.e., dragosine), methyl gentisate, 1,2-hexandiol and 1,2-octandiol (i.e., combination sold as Symdiol 68 by Symrise AG, Germany), inositol, decylenoylphenylalanine (e.g., sold under the tradename Sepiwhite by Seppic, France), kojic acid, hexamidine compounds, salicylic acid, and retinoids including retinol and retinyl propionate.

In certain embodiments, the additional skin tone agent is selected from vitamin B3 compounds, sugar amines, hexamidine compounds, salicylic acid, 1,3-dihydroxy-4-alkylbenzene such as hexylresorcinol, and retinoids. As used herein, “vitamin B₃ compound” means a compound having the formula:

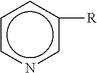

wherein R is —CONH₂ (i.e., niacinamide), —COOH (i.e., nicotinic acid) or —CH₂OH (i.e., nicotinyl alcohol); derivatives thereof; and salts of any of the foregoing. As used herein, “sugar amine” includes isomers and tautomers of such and its salts (e.g., HCl salt) and its derivatives. Examples of sugar amines include glucosamine, N-acetyl glucosamine, mannosamine, N-acetyl mannosamine, galactosamine, N-acetyl galactosamine, their isomers (e.g., stereoisomers), and their salts (e.g., HCl salt). As used herein, “hexaminide compound” means a compound having the formula:

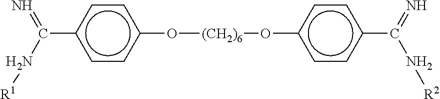

wherein R¹ and R² are optional or are organic acids (e.g., sulfonic acids, etc.). In one embodiment, hexamidine compound is hexamidine diisethionate.

2. Anti-Inflammatory Agents

The composition may additionally include an anti-inflammatory agent. When present, the compositions herein may contain up to about 20%, 10%, 5%, 3%, or 1% by weight based on the weight of the composition, of the anti-inflammatory agent. When present, the compositions herein may contain at least about 0.001%, 0.01%, 0.1%, 0.2%, 0.3%, 0.5%, or 1% by weight, based on the weight of the composition, of the anti-inflammatory agent. Suitable ranges include any combination of the lower and upper limits Suitable anti-inflammatory agents include, but are not limited to nonsteroidal anti-inflammatory agents (NSAIDS including but not limited to ibuprofen, naproxen, flufenamic acid, etofenamate, aspirin, mefenamic acid, meclofenamic acid, piroxicam and felbinac), glycyrrhizic acid (also known as glycyrrhizin, glycyrrhixinic acid, and glycyrrhetinic acid glycoside) and salts such as dipotassium glycyrrhizate, glycyrrhetenic acid, licorice extracts, bisabolol (e.g., alpha bisabolol), manjistha (extracted from plants in the genus Rubia, particularly Rubia cordifolia), and guggal (extracted from plants in the genus Commiphora, particularly Commiphora mukul), kola extract, chamomile, red clover extract, and sea whip extract (extracts from plant in the order Gorgonacea), derivatives of any of the foregoing, and mixtures thereof.

3. Sunscreen Actives

The compositions of the subject invention may comprise one or more sunscreen actives (or sunscreen agents) and/or ultraviolet light absorbers. Herein, “sunscreen active” collectively includes, sunscreen actives, sunscreen agents, and/or ultraviolet light absorbers. Sunscreen actives include both sunscreen agents and physical sunblocks. Sunscreen actives may be organic or inorganic. Examples of suitable sunscreen actives are disclosed in Personal Care Product Council's International Cosmetic Ingredient Dictionary and Handbook, Thirteenth Edition, as “sunscreen agents.” Particularly suitable sunscreen actives are 2-ethylhexyl-p-methoxycinnamate (commercially available as PARSOL™ MCX), 4,4′-t-butyl methoxydibenzoyl-methane (commercially available as PARSOL™ 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, menthyl anthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, benzylidene camphor and derivatives thereof, titanium dioxide, and mixtures thereof.

In one embodiment, the composition may comprise from about 1% to about 20%, and alternatively from about 2% to about 10% by weight of the composition, of the sunscreen active. Exact amounts will vary depending upon the chosen sunscreen active and the desired Sun Protection Factor (SPF), which is within the knowledge of one of skilled in the art.

C. Dermatologically Acceptable Carrier

The compositions herein may also comprise a dermatologically acceptable carrier (which may be referred to as “carrier”) for the composition. The phrase “dermatologically acceptable carrier”, as used herein, means that the carrier is suitable for topical application to the skin, has good aesthetic properties, is compatible with the actives in the composition, and will not cause any unreasonable safety or toxicity concerns. In one embodiment, the carrier is present at a level of from about 50% to about 99%, about 60% to about 98%, about 70% to about 98%, or, alternatively, from about 80% to about 95%, by weight of the composition.

The carrier can be in a wide variety of forms. Non-limiting examples include simple solutions (e.g., aqueous, organic solvent, or oil based), emulsions, and solid forms (e.g., gels, sticks, flowable solids, or amorphous materials). In certain embodiments, the dermatologically acceptable carrier is in the form of an emulsion. Emulsion may be generally classified as having a continuous aqueous phase (e.g., oil-in-water and water-in-oil-in-water) or a continuous oil phase (e.g., water-in-oil and oil-in-water-in-oil). The oil phase of the present invention may comprise silicone oils, non-silicone oils such as hydrocarbon oils, esters, ethers, and the like, and mixtures thereof.

The aqueous phase typically comprises water. However, in other embodiments, the aqueous phase may comprise components other than water, including but not limited to water-soluble moisturizing agents, conditioning agents, anti-microbials, humectants and/or other water-soluble skin care actives. In one embodiment, the non-water component of the composition comprises a humectant such as glycerin and/or other polyols. However, it should be recognized that the composition may be substantially (i.e., less than 1% water) or fully anhydrous.

A suitable carrier is selected to yield a desired product form. Furthermore, the solubility or dispersibility of the components (e.g., carob fruit extract, sunscreen active, additional components) may dictate the form and character of the carrier. In one embodiment, an oil-in-water or water-in-oil emulsion is preferred.

Emulsions may further comprise an emulsifier. The composition may comprise any suitable percentage of emulsifier to sufficiently emulsify the carrier. Suitable weight ranges include from about 0.1% to about 10% or about 0.2% to about 5% of an emulsifier by weight based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986). Suitable emulsions may have a wide range of viscosities, depending on the desired product form.

The carrier may further comprise a thickening agent as are well known in the art to provide compositions having a suitable viscosity and rheological character.

### II. Exemplary Compositions

Table 1 sets forth non-limiting examples of compositions suitable for use herein. The examples are given solely for the purpose of illustration and are not to be construed as limitations of the present invention, as many variations thereof are possible without departing from the spirit and scope of the invention, which would be appreciated by one of ordinary skill in the art. The listed formulations comprise the listed components and any minor materials associated with such components (e.g., filler or diluents), which may vary depending on the physical and chemical characteristics of the particular ingredients selected to make the composition.

All Examples may be used to improve the appearance of one or more areas of facial pores. The In Vivo product illustrated in Table 1 was the product used in the In Vivo testing described in more detail below.

TABLE 1

| | | | | | | In Vivo |
|---|---|---|---|---|---|---|
| Component | Ex. A | Ex. B | Ex. C | Ex. D | Ex. E | Product |
| Carob fruit extract *1 | 3.000 | 1.000 | 1.000 | 0.550 | 6.000 | 3.000 |
| N-Acetylglucosamine | 0 | 0 | 2.000 | 0 | 0 | 0 |
| Hexamidine Diisethionate | 0 | | | 0.090 | 0.090 | |
| Undecylenoyl- | 0 | 1.000 | 0.500 | 0 | 0 | 0 |
| phenylalanine *2 | | | | | | |
| (neutralized) | | | | | | |
| Dipotassium Glycyrrhizate | 0 | 0.300 | 0.100 | 0.100 | 0.100 | 0 |
| Niacinamide | 5.000 | 5.000 | 5.000 | 5.000 | 5.000 | 0 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Cetearyl glucoside + | 0.200 | 0.200 | 0.200 | 0.200 | 0.200 | 0.200 |
| cetearyl alcohol *3 | | | | | | |
| Behenyl alcohol | 0.400 | 0.400 | 0.400 | 0.400 | 0.400 | 0.400 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 3.000 |
| Polyacrylamide + C13-14 | 2.000 | 2.000 | 2.000 | 2.000 | 2.000 | 2.000 |
| isoparaffin + laureth-7 *4 | | | | | | |
| Disodium EDTA | 0.100 | 0.100 | 0.100 | 0.100 | 0.100 | 0.3 |
| Benzyl alcohol | 0.400 | 0.400 | 0.400 | 0.400 | 0.400 | 0.400 |
| Dimethicone/ | 2.000 | 2.000 | 2.000 | 2.000 | 2.000 | 2.000 |
| Dimethiconol *5 | | | | | | |
| Homosalate | 0 | 0 | 0 | 0 | 9.000 | 0 |
| Avobenzone | 0 | 0 | 0 | 0 | 3.000 | 0 |
| Octocrylene | 0 | 0 | 0 | 0 | 2.600 | 0 |
| Oxybenzone | 0 | 0 | 0 | 0 | 1.000 | 0 |
| Octisalate | 0 | 0 | 0 | 0 | 4.500 | 0 |
| Water | QS | QS | QS | QS | QS | QS |
| TOTAL | 100 | 100 | 100 | 100 | 100 | 100 |

*1 - Glyco-Repair ™PX, available from Silab S.A.
*2 - Sepiwhite available from SEPPIC, France.
*3 - Emulgade PL 68/50 available from Cognis GmbH.
*4 - Sepigel 305, available from SEPPIC, France.
*5 - Dow Corning DC1503 available from Dow Corning, Inc., Midland, MI.

The compositions of the present invention are generally prepared by conventional methods such as are known in the art of making topical compositions. Such methods typically involve mixing of the ingredients in one or more steps to a relatively uniform state, with or without heating, cooling, application of vacuum, and the like. Typically, emulsions are prepared by first mixing the aqueous phase materials separately from the fatty phase materials and then combining the two phases as appropriate to yield the desired continuous phase. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

### III. Methods of Treatment

Various methods of treatment, application, regulation, or improvement may utilize the aforementioned compositions. In one embodiment, the method includes the step of identifying facial pores for improvement by the composition. The facial pores may be identified by the user or a third party such as a dermatologist, cosmetician, or other caregiver. Identification may be achieved by visual inspection of the skin for facial pores in need of treatment based on appearance. Identification may also be achieved by commercially available imaging devices such as the VISIA® Complexion Analysis system (available from Canfield Scientific, Inc., Fairfield, N.J.). This device is capable of collecting images of the skin and identifying facial pores. In some instances, the method comprises the step of identifying a plurality of facial pores areas for treatment by the composition. Identification of the facial pores may occur on any facial skin surface, including the forehead, perioral, chin, periorbital, nose, and/or cheek skin surfaces. In some embodiments, the facial pores of the nose and cheek skin surfaces may be targeted.

The method may comprise the step of applying the composition to a facial skin surface, which may have been previously identified as having facial pores in need of treatment. Many regimens exist for the application of the composition to the facial pores. The composition may be applied at least once a day, twice a day, or on a more frequent daily basis, during a treatment period. When applied twice daily, the first and second applications are separated by at least 1 to about 12 hours. Typically, the composition may be applied in the morning and/or in the evening before bed.

The treatment period is ideally of sufficient time to provide an improvement in the appearance of the facial pores. The treatment period may be at least about 1 week. The treatment period may last about 4 weeks or about 8 weeks. In certain embodiments, the treatment period will extend over multiple months (i.e., 3-12 months) or multiple years. In one embodiment the composition is applied to the facial pores at least once a day during a treatment period of at least about 4 weeks or at least about 8 weeks. In one embodiment the composition is applied to the facial pores twice a day during a treatment period of at least about 4 weeks or 8 weeks.

The step of applying the composition to the facial pores may be performed by localized application. In reference to application of the composition, the term “localized”, “local”, or “locally” mean that the composition is delivered to the targeted area (such as the region of facial pores) while minimizing delivery to skin surface not requiring treatment. The composition may be applied and lightly massaged into the facial pores. It is recognized that localized application does allow for a reasonable amount of the composition to be applied to areas adjacent the facial pores (i.e., the composition is unlikely to be applied or to remain within the boundary of the facial pores without some spreading). The form of the composition or the dermatologically acceptable carrier should be selected to facilitate localized application. While certain embodiments of the present invention contemplate applying a composition locally to facial pores, it will be appreciated that compositions herein can be applied more generally or broadly to one or more facial skin surfaces to reduce the appearance of facial pores within those facial skin regions.

In some embodiments, the composition may be delivered by a variety of applicators appropriate for localized and general application. In another embodiment, the composition is applied to the one or more facial pores regions and more generally to one or more facial skin surfaces contemporaneously (i.e., over a period of less than 30 minutes or, more typically, less than 5 minutes). While some methods described herein contemplate applying the compositions herein with an applicator, it will be appreciated that applicators are not required and the compositions can also be applied directly by using a finger or in other conventional manners.

For general application to a skin surface and, particularly a facial skin surface, the dosed amount of the composition may be between about 1 to about 50 uL/cm² per application (i.e., per single application to the skin surfaces).

One suitable method of improving the appearance of facial pores includes the step of topically applying a composition comprising an effective amount of carob fruit extract to the facial pores on a skin surface, wherein the composition is applied for a period of time sufficient for carob fruit extract to improve the appearance of the facial pores. Another suitable method of improving the appearance of facial pores includes the steps of first identifying facial pores on a skin surface, applying a composition comprising an effective amount of carob fruit extract to the facial pores on the skin surface, wherein the composition is applied for a period of time sufficient for carob fruit extract to improve the appearance of the facial pores.

### VI. In Vivo Testing

A 12 week in vivo study was conducted to evaluate the effect of carob fruit extract on overall skin micro-texture. Skin micro-texture is highly correlated to the appearance of enlarged pores, and thus an improvement in skin micro-texture is generally indicative of an improvement in enlarged pore appearance (i.e., smaller pores).

Skin “micro-texture” refers to facial skin surface features that are smaller in size than traditional fine lines and wrinkles (i.e., “macro-texture”). Micro-textural features are generally depressions less than 5 mm in length and thinner than 0.16 mm in breadth (i.e., features larger than those which are generally referred to as “fine lines and wrinkles” or “macro-texture”). Micro-textured skin is often described as having a “pebbled” appearance, with a bumpy visual texture similar to that of an orange peel (i.e., peel of the Citrus sinensis fruit).

The test formulations included a control composition (i.e., the vehicle only) and a test composition (i.e., the vehicle+3.00% Glyco-Repair™PX carob fruit extract). The test composition is illustrated in Table 1 above as the “In Vivo Product.” Improvement in the appearance of facial texture was measured by expert visual grading of high-resolution digital images taken at baseline (i.e., prior to treatment) and at 4, 8 and 12 weeks using the Rapid Evaluation of Anti-aging Leads (REAL 3.0) system. The REAL system and its use are described in “A randomized, controlled comparative study of the wrinkle reduction benefits . . . ” J. J. J. Fu et al., British Journal of Dermatology, Vol. 162, 2010, pp. 647-654. Three trained expert graders independently assessed changes in the appearance of facial texture on the cheeks by comparing baseline and post-treatment images side-by-side using a ±8-point ordinal scale. The three trained expert graders also independently assessed changes in the appearance of facial texture on the cheeks by comparing the post-treatment images to control images using a ±8-point ordinal scale. The control images are images taken of a portion of the face of the test subject prior to treatment. The expert graders and other assessors were blinded to the treatments. The area considered in the facial texture assessment includes the cheek area of the face that is below the eye socket and above the bottom of the jaw line extending laterally from the side of the nose straight down across the outer portion of the mouth and lips down to the chin, extending across the cheek to the ear. The grading area does not include the outer edge of the cheek and jaw line.

Table 2 illustrates the change in facial appearance on the cheeks as determined by the expert graders. As illustrated in Table 2, at weeks 4, 8, and 12, the facial texture associated with the test composition was found to be improved versus the control and baseline values, as indicated by a positive fold-increase in micro-texture improvement (i.e., a positive Mean Change From Control value and Mean Change From Baseline value).

TABLE 2
Improvement in Skin Micro-Texture

| Treatment | | Mean Change | | Mean Change | |
|---|---|---|---|---|---|
| Phase | N | From Control | P-value | from Baseline | P-value |
| Week 4 | 65 | 0.293 | 0.0575* | 0.440 | 0.0057* |
| Week 8 | 66 | 0.116 | 0.2740 | 0.497 | 0.0022* |
| Week 12 | 64 | 0.191 | 0.1747+ | 0.222 | 0.1963+ |

*= statistically significant
+= trending

### VII. In Vitro Testing

An in vitro skin biomarker test was conducted to demonstrate the effect of carob seed extract on a cultured human skin model. The model used in the test is a MatTek™ brand human skin equivalent available from the MatTek Corporation, which is a full thickness culture system that emulates normal human skin properties and function. The MatTek™ brand skin model contains a three-dimensional, highly differentiated human epidermis with 8-12 cell layers including basal, spinous, granular and stratum corneum layers. The epidermis is grown above a human dermal fibroblast-containing collagen matrix. The skin model is configured to permit the topical application of test materials to the stratum corneum surface of the model. The skin model cultures are supplied in 24-well plate. Each well of the plate includes a snap-well fitting and the skin culture in a media-supplemented agarose gel.

Prior to testing, the skin model cultures are examined for visual defects and equilibrated overnight at 37° C., 5% CO₂, and 95% RH. The skin cultures were transferred to fresh 24-well plates by removing the snap-well fitting along with the skin culture and placing it in a well of the new plate. Each well of the new 2 mL/well of fresh pre-warmed assay maintenance media (available from the MatTek Corporation as part of the EpiDermFT™ kit) in the bottom of each well. A first set of six skin culture samples were treated with Dulbecco's phosphate buffered saline (“DPBS”) by topically applying 40 μL of DPBS to the stratum corneum surface of each skin culture sample. The DPBS was used as a vehicle control. DPBS is available from the MatTek Corporation as part of the EpiDermFT™ kit. A second set of six skin culture samples were treated with a 2 ng/mL solution of transforming growth factor beta (“TGF-b”) by adding a stock solution of TGF-b to the media of the wells at a dilution of 1:1000. The TGF-b was used as a positive control due to its known effect of inducing the production of procollagen I and hyalauronic acid in skin tissue. The TGF-b stock solution was prepared by reconstituting powdered TGF-b in water containing 0.1% Bovine Serum Albumin (“BSA”), to a concentration of 2 ug/mL. The stock solution was stored in working aliquots at −20° C. A final set of six skin culture samples were treated with 3% carob seed extract (97% DPBS) solution by topically applying the carob seed extract solution to the stratum corneum surface of the skin culture samples. After treatment, the skin cultures were incubated at 37° C., 5% CO₂, and 95% RH for 24 hours.

Twenty-four hours after treatment, the skin cultures were removed from the incubator and visually inspected for defects (e.g., to see if media leaked up through the culture insert). Visibly damaged or defective cultures are discarded. In this test, two cultures treated with the DPBS were discarded and one culture treated with TGF was discarded. In addition, the viability of the cultures was confirmed with an MTT test, which is a commonly known method of measuring the activity of certain cellular enzymes. The MTT test was conducted using an MTT-200 kit, available from the MatTek Corporation. Next, a 5 mm punch biopsy sample was taken from each of the remaining skin cultures. Each biopsy sample was transferred to a 1.5 mL centrifuge tube and 500 μL of a mild tissue protein extraction reagent (“T-per”) designed for total protein extraction was added to each tube. In addition, a 3 mm tungsten bead was added to the tube to aid in subsequent homogenization. The cultures were placed on ice for 30 minutes. The chilled cultures were homogenized using a Qiagen™ brand mixer mill at full speed (˜30 shakes/sec) for 3 min, rotated and then homogenized an additional 3 min. The homogenized samples were centrifuged at 10,000 rpm for 15 minutes. The supernatant was collected and analyzed with a Micro BCA kit to quantitate protein concentration. The extracted proteins were run through a series of biomarker evaluations using a commercially available Pierce Micro BCA Protein Assay in combination with a Spectrafluou Plus™ brand spectrophotometer. The amount of procollagen I and hyalauronic acid present in the test cultures were measured using commercially available ELISA kits (e.g., from Takara Bio, Inc. and Corgenix).

Procollagen I was selected because it is a known precursor to collagen, which is an important component of healthy skin. Hyaluronic acid was selected because it serves several important physiologic functions related to skin health, including, for example, barrier effects, cell proliferation and migration, tissue resiliency and elasticity, wound healing, and overall skin hydration. The skin health benefits associated with increased procollagen I and hyalauronic acid production can ultimately lead to firmer skin, which may cause a reduction in pore size and improved skin appearance. The results of the test are illustrated in Tables 3 and 4 below. Tables 3 and 4 illustrates the optical density (“OD”), amount of protein of interest (i.e., procollagen I or hyalauronic acid), and amount of total protein after treatment of the skin cultures with each of the three test compositions (i.e., DPBS, TGF-b and carob seed extract). In addition, Tables 3 and 4 also show the normalized protein value and the average normalized protein value. The normalized protein value is determined by dividing the amount of procollagen I or hyalauronic acid in a well by the total protein in the well. A one-sided P-value was used to show the statistical significance of the results.

TABLE 3
Procollagen I

| | | Procol- | Pro- | Normal- | Avg Nor- | |
|---|---|---|---|---|---|---|
| | | lagen I | tein | ized | malized | P- |
| | OD | (ng/mL) | (μg) | protein | Protein | value |
| DPBS | 0.2854 | 44 | 4 | 13 | 13.3 | 1.00 |
| | 0.3555 | 59 | 5 | 12 | | |
| | 0.2036 | 28 | 2 | 14 | | |
| | 0.2950 | 47 | 3 | 15 | | |
| 2 ng/mL TGF-b | 0.8378 | 159 | 2 | 86 | 29.8 | 0.04 |
| (positive ctrl) | 0.5297 | 96 | 2 | 47 | | |
| | 0.4262 | 74 | 3 | 24 | | |
| | 0.4376 | 77 | 3 | 28 | | |
| | 0.3183 | 52 | 3 | 20 | | |
| 3% Glyco-Repair | 0.4373 | 76 | 2 | 39 | 47.2 | 0.02 |
| | 0.6252 | 116 | 2 | 56 | | |
| | 0.4324 | 76 | 3 | 25 | | |
| | 0.5759 | 104 | 2 | 44 | | |
| | 0.5079 | 91 | 3 | 34 | | |
| | 0.9307 | 178 | 2 | 86 | | |

TABLE 4
Hyalauronic Acid

| | | | Pro- | Normal- | | |
|---|---|---|---|---|---|---|
| | | HA | tein | ized | | P- |
| | OD | (ng/mL) | (ng) | protein | Avg | value |
| DPBS | 1.2494 | 613 | 293 | 13 | 2.0 | 1.00 |
| | 0.9343 | 446 | 411 | 12 | | |
| | 0.9349 | 446 | 171 | 14 | | |
| | 1.1866 | 580 | 251 | 15 | | |
| 2 ng/mL TGF-b | 1.1523 | 562 | 153 | 86 | 2.8 | 0.10 |
| (positive ctrl) | 0.9989 | 480 | 166 | 47 | | |
| | 1.22 | 598 | 257 | 24 | | |
| | 1.1177 | 545 | 228 | 28 | | |
| | 1.1639 | 568 | 215 | 20 | | |
| 3% Glyco-Repair | 1.0596 | 513 | 161 | 39 | 2.8 | 0.04 |
| | 1.1059 | 538 | 171 | 56 | | |
| | 1.2051 | 590 | 251 | 25 | | |
| | 1.1151 | 543 | 195 | 44 | | |
| | 1.0715 | 520 | 220 | 34 | | |
| | 1.095 | 532 | 171 | 86 | | |

From Table 3 it can be seen that the 3% carob seed extract resulted in substantially more procollagen I production relative to both the vehicle control and the positive control. From Table 4, it can be seen that the 3% carob seed extract resulted in substantially more hyalauronic acid production relative to the vehicle control and approximately the same amount of hyalauronic acid production as the positive control. Inducing as much as hyalauronic acid production as TGF-b is a significant benchmark because TGF-b is known to play a role in procollagen I and hyalauronic acid production in the body. However, TGF-b does not make a suitable skin active agent due to its known cancer cell proliferation effects when topically applied to skin. Thus, identifying agents with similar or superior procollagen I and hyalauronic acid producing effects as TGF-b but without its drawbacks is important for providing improved skin care compositions. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of improving the appearance of facial pores, comprising:
a. identifying a region of facial pores on a facial skin surface in need of treatment; and
b. applying a composition comprising an effective amount of carob fruit extract to the facial pores on the facial skin surface, wherein the composition is applied for a period of time sufficient to improve the appearance of the facial pores.

2. The method of claim 1, wherein the composition has a concentration of carob fruit extract of from 0.0001 wt % to 15 wt % based on the weight of the composition.

3. The method of claim 1, wherein the composition is applied to at least one facial skin surface selected from the group consisting of a forehead, perioral, chin, periorbital, nose, cheek skin surface, and combinations thereof.

4. The method of claim 1, wherein the composition is applied to the facial skin surface at least once a day for at least four weeks.

5. The method of claim 4, wherein the composition is applied to the facial skin surface at least twice a day for at least eight weeks.

6. The method of claim 1, wherein a diameter of at least some of the facial pores is decreased.

7. The method of claim 1, wherein the composition further comprises a sunscreen active.

8. The method of claim 1, wherein the composition further comprises an anti-inflammatory agent.

9. The method of claim 8, wherein the anti-inflammatory agent is selected from the group consisting of glycyrrhizic acid, glycyrrhizic acid salts, licorice extract, bisabolol, and combinations thereof.

10. The method of claim 1, wherein the composition further comprises a skin tone agent.

11. The method of claim 10, wherein the skin tone agent is selected from the group consisting of vitamin B3 compounds, sugar amines, hexamidine compounds, salicylic acid, 1,3-dihydroxy-4-alkylbenzene, retinoids, and combinations thereof.

12. The method of claim 1, wherein the step of identifying facial pores is performed by an imaging device.

13. The method of claim 1, wherein the step of identifying facial pores is performed visually by the human eye.

14. The method of claim 1, wherein the composition is applied using an applicator.

15. The method of claim 1, wherein the composition is applied to more than one facial skin surface contemporaneously.
